(19) United States
(12) Patent Application Publication
(10) Pub. No.: US 2005/0096509 A1
(43) Pub. Date: May 5, 2005

(54) Nanotube treatments for internal medical devices

(76) Inventor: Greg Olson, Elk River, MN (US)

Correspondence Address: KENYON & KENYON, 1500 K STREET, N.W., SUITE 700, WASHINGTON, DC, 20005, US

(21) Appl. No.: 10/699,694

(22) Filed: Nov. 4, 2003

(51) Int. Cl. A61M 25/00

(52) U.S. Cl. 600/300; 604/265; 427/2.1; 604/500

(57) ABSTRACT

Nanotube treatments for internal medical devices are provided in the present invention. This may include a medical apparatus sized for insertion into a patient wherein the medical apparatus has a plurality of nanotubes associated with one of its surface. This invention may also include a diagnostic method that comprises inserting a plurality of nanotubes into a body of a patient, positioning the plurality of nanotubes at a target site within the body of the patient, interfacing the plurality of nanotubes with the target site, removing the plurality of nanotubes from the target site, and analyzing the plurality of nanotubes after they have been removed from the target site. This invention may also include a method for manufacturing a medical device sized for insertion into the body. The method comprising providing a medical device and interfacing the medical device with a plurality of nanotubes.

### FIELD OF THE INVENTION

The present invention is directed toward using nanotubes as a coating or surface treatment for medical devices that may be used within the body of a patient. More specifically, the present invention is directed toward positioning or placing nanotubes on at least one surface of a medical device to enhance the performance, diagnostic capabilities or usefulness of the medical device. The nanotubes, in some embodiments, may be pretreated or interfaced with a therapeutic, a carrier of some kind or both.

### BACKGROUND

Nanotubes are tube-like single wall or multi-wall structures, most often composed of carbon, that typically measure a few nanometers in width and several nanometers or even centimeters in length. When made from carbon, they can behave like metals or semiconductors, can conduct electricity better than copper, can transmit heat better than diamond, and rank among the strongest materials known.

Invasive medical procedures are medical procedures wherein a practitioner will physically invade the body of a patient in order to diagnose or treat the patient. These procedures range from highly invasive procedures such as open heart surgery to minimally invasive procedures such as balloon angioplasty or endoscopic surgery. During each of these procedures a practitioner will temporarily or permanently insert or place medical devices within the body of the patient to carry out the procedure. These medical devices may be used to make physical alterations within the body and to sample target areas within the body for further diagnosis or analysis. Typical medical devices used for these purposes include delivery catheters, suction catheters, and medical implants, such as stents.

### BRIEF DESCRIPTION

Nanotube treatments for internal medical devices are provided in the various embodiments of the present invention. In one embodiment, a medical apparatus is provided. This apparatus may be sized for insertion into a patient and may have a plurality of nanotubes associated with one of its surface. In another embodiment, a diagnostic method is provided. This method may include inserting a plurality of nanotubes into a body of a patient, positioning the plurality of nanotubes at a target site within the body of the patient, interfacing the plurality of nanotubes with the target site, removing the plurality of nanotubes from the target site, and analyzing the plurality of nanotubes after they have been removed from the target site. In another embodiment, a method for manufacturing a medical device sized for insertion into the body may be provided. This method may include providing a medical device and interfacing a medical device with a plurality of nanotubes.

### BRIEF DESCRIPTION OF THE DRAWINGS

FIG. 1 is a flow chart of a method that may be used in accord with an embodiment of the present invention.

FIG. 2 is a side sectional view of a treated medical implant in accord with an alternative embodiment of the present invention.

FIG. 3 is a side sectional view of a treated medical implant in accord with an alternative embodiment of the present invention.

FIG. 4 is a side view of a nanotube in accord with an alternative embodiment of the present invention.

FIG. 5 is a side view of a nanotube in accord with an alternative embodiment of the present invention.

FIG. 6 is a side view of a broken nanotube in accord with an alternative embodiment of the present invention.

FIG. 7 is a side view of steps that may be taken to perform a diagnostic procedure in accord with an alternative embodiment of the present invention.

FIG. 8 is a side view of a treated medical implant in accord with an alternative embodiment of the present invention.

### DETAILED DESCRIPTION

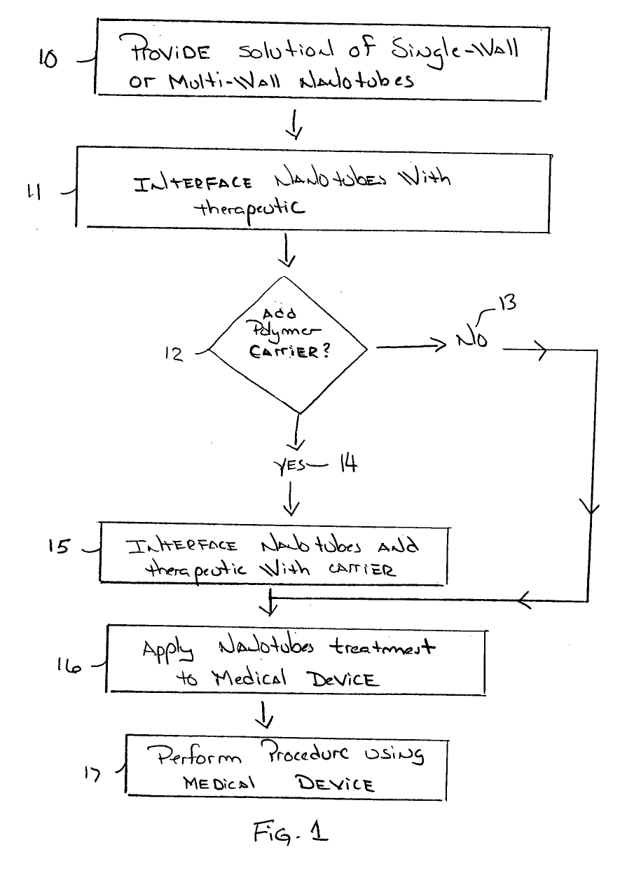

The present invention is directed towards the use of nanotubes in various devices, systems, and medical procedures. FIG. 1 is a flow chart directed to an embodiment of the present invention. In the process depicted by the flow chart of FIG. 1, a medical device, which has been interfaced with single wall or multi-wall nanotubes, is used to perform a medical procedure. In this embodiment, a solution of single wall or multi-wall nanotubes should be provided as indicated in step 10. In a preferred embodiment, these nanotubes will be carbon nanotubes but other materials may be used as well. These other materials may include nucleotides, guamine and systosine. Once provided, the nanotubes may be interfaced with a preselected therapeutic as indicated in step 11. Then, if a polymer carrier is to be added, the nanotubes and therapeutic may be interfaced with the carrier. This is shown in step 15. In some instances, the nanotubes and therapeutic may need to be taken out of solution prior to interfacing them with the carrier while in others this may not be necessary. Conversely, if no carrier is to be used, step 15 is skipped. Then, at step 16, the therapeutic and nanotubes (and in some instances the carrier as well) may be applied or otherwise interfaced with the medical device. In so doing, a layer of nanotubes may be formed on a surface of the device.

The medical devices that may be used in this and other embodiments include stents, vena cava filters, aneurism coils, catheters, and injection devices. Applying or otherwise interfacing the nanotubes and therapeutic to the medical device may include submerging the medical device in a vessel of nanotubes and therapeutic, spraying the nanotubes and therapeutic onto the medical device or using some other application method. In addition, in this and other embodiments the nanotubes may cover the entire device or only a portion of the device. Once the medical device is treated, it may then be used, as indicated in step 17, to perform a medical procedure.

FIG. 2 is a side sectional view of a treated insertable medical device in accord with an alternative embodiment of the present invention. The treated insertable medical device 20 in this embodiment includes an outside surface 25, an inside surface 24, an internal channel 26, and a wall or strut 23. In this embodiment, the outside surface 25 of the wall 23 of the device 20 has been coated with a single layer of nanotubes and therapeutic while the inside surface 24 of the wall 23 has been treated with more than a single layer of nanotubes and therapeutic. The nanotubes and therapeutic in this embodiment have been interfaced with one another without the benefit of a carrier. Thus, the nanotubes are not within a polymer or other carrier as in other embodiments.

While the outside surface 25 of the medical device 20 in FIG. 2 has a single layer of nanotubes and therapeutic, in alternative embodiments this surface may not be treated at all or may have more than a single layer of nanotubes and therapeutic or a layer of nanotubes and a layer of coating. Likewise, in other alternative embodiments, the inside surface, which is shown with more than one layer of nanotubes and therapeutic, may instead contain only a single layer of nanotubes and therapeutic, a layer of nanotubes and a layer of coating or no treatment at all. As indicated above, the medical device in this and other embodiments may include stents, vena cava filters, aneurism coils, catheters, and injection devices.

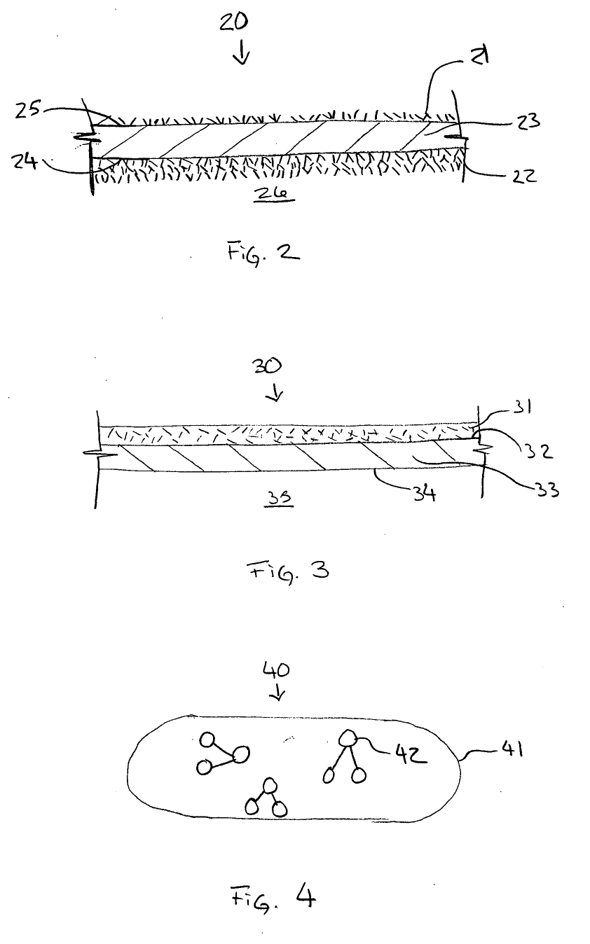

FIG. 3 is a side view of a treated insertable medical device 30 in accord with another alternative embodiment of the present invention. In this embodiment, the device 30 has a wall or strut 33 with an outside surface 32 wherein the wall 33 helps to define an internal channel or lumen 35 as would be found in a stent or a catheter. In this embodiment, the nanotubes and therapeutic are positioned solely on the outside surface 32 of the device. In addition, the nanotubes and therapeutic are contained within a polymer carrier rather than simply being interfaced solely with one another as described in the proceeding embodiment. The polymer carrier in this embodiment may contain more than a single layer of nanotubes and therapeutic and these nanotubes and therapeutic may be homogenously or randomly positioned throughout the polymer carrier.

FIG. 4 is a side view of a single wall nanotube delivery system in accord with another alternative embodiment of the present invention. In FIG. 4, the nanotube delivery system 40 consists of a nanotube cage 41 and therapeutic molecule 42 contained within the nanotube cage 41. In this embodiment the nanotube cage 41 has been sized to contain an entire therapeutic molecule 42. This molecule may then be carried by the nanotube cage 41 and may be released at a target site once the nanotube is delivered and positioned near the target site.

The nanotube delivery system 40 of FIG. 4 may be created once the nanotubes and therapeutic are interfaced with one another as described in the embodiment of FIG. 1. Once created, this nanotube delivery system may be used to coat or cover a medical device that will be placed in the body. Once in the body, the therapeutic molecule 42 may be released from the nanotube delivery system 40 to the surrounding area. Alternatively, the therapeutic may remain within the nanotube 41 until the nanotube is dissolved or otherwise broken down or apart.

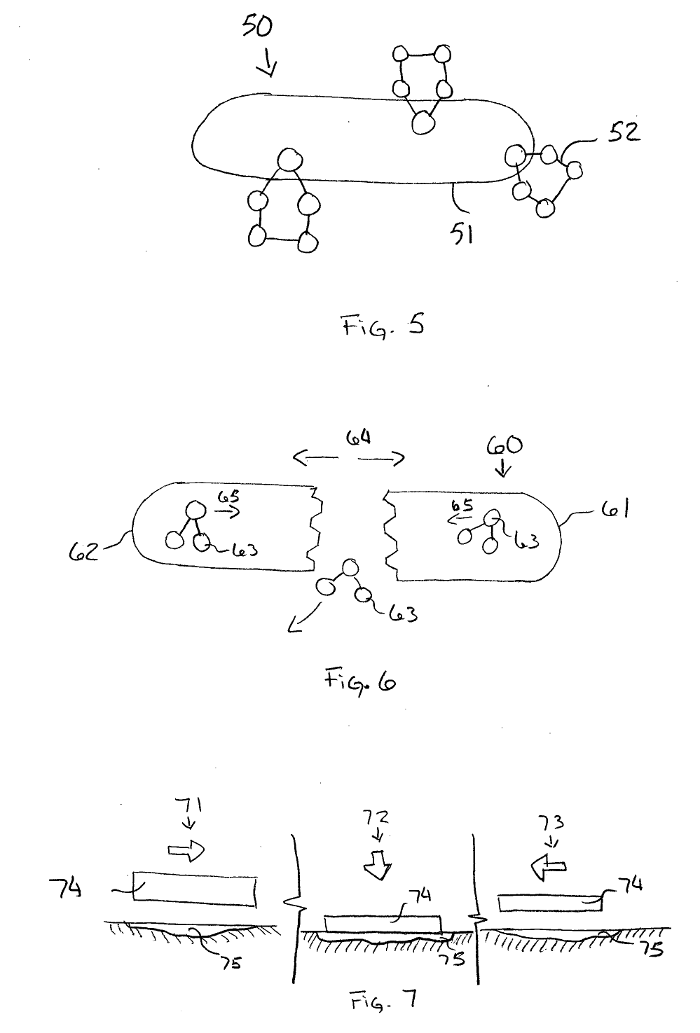

FIG. 5 is a side view of a nanotube delivery system in accord with another alternative embodiment of the present invention. In FIG. 5, rather than containing an entire therapeutic molecule 52 within the nanotube 51, as in FIG. 4, the therapeutic 52 is only partially retained within the nanotube 51. Thus, as can be seen, a portion of the therapeutic molecule 52 is within the nanotube 51 while a relatively larger portion of the therapeutic molecule 52 is outside of the nanotube 51.

Alternatively, in another alternative embodiment, the entire therapeutic molecule may be outside of the nanotube. In this embodiment, chemical or other forces may be used to associate or adhere the nanotubes to the therapeutic. Then, once the nanotubes reach a delivery site, the chemical or other bonds that associate the nanotubes to the therapeutic may be broken when the therapeutic is delivered to the target site.

FIG. 6 is a side view of a nanotube delivery system 60 in accord with another alternative embodiment of the present invention. In this embodiment, the nanotube, which contains therapeutic 63 within it, has been broken into halves 61 and 62. Arrows 64 indicate the direction in which the nanotube has been cleaved apart. Once broken, the therapeutic 63 within the nanotube moves out of the nanotube as indicated by arrows 65. Thus, in this embodiment, the nanotube is sized in relation to the therapeutic to act as a cage and retain the therapeutic within it. Then, when forces placed on the nanotube exceed its structural tolerances, the nanotube breaks and therapeutic within it is released to the surrounding area.

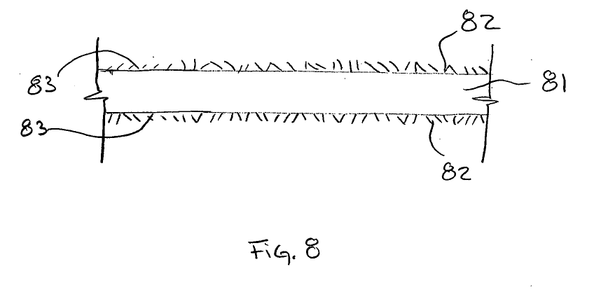

FIG. 7 is an another alternative embodiment of the present invention. In FIG. 7 a diagnostic method is provided. In this embodiment a nanotube diagnostic 74 is positioned near a target area 75 (as indicated by arrow number 71), the nanotube diagnostic 74 is then urged against the target area 75 (as indicated by arrow 72). Then, once the nanotube diagnostic 74 has been exposed to the target area 75, it is withdrawn from the vicinity of the target area 75 in order to be tested and analyzed. In so doing, the nanotube diagnostic 74 is exposed to a target area so that it may sample, absorb, or mimic the contours of the target area. After being exposed to the target area, the nanotube diagnostic may be sampled, analyzed or studied in order to diagnose the state, composition or shape of the target area.

In one embodiment, the nanotube diagnostic 74 may be the distal end of a balloon catheter that has been covered with single wall carbon nanotubes. These nanotubes may then be pressed towards or into the target area 75, which may be a suspected cancerous tumor or other abnormality, while the nanotubes are near or are in contact with the tumor they may absorb, grasp or attract portions of the tumor. Then, with the portions of the tumor coupled to it, the balloon catheter may be removed and its distal end, containing the nanotubes and its samples, may be analyzed and studied in order to better understand and diagnose the target area. Likewise, the nanotubes may conform to the target area such that the profile obtained may be analyzed in order to better understand and diagnose the target area. Still further, samples of the target area may adhere to the nanotubes and may be removed from the target area to be analyzed.

FIG. 8 is a side view of a catheter treated with nanotubes in accord with another alternative embodiment of the present invention. In this embodiment the external surface 83 of the catheter 81 has been treated and covered with nanotubes 82. This layer of nanotubes may cover the entire exterior portion of the catheter or only a section of it. The nanotubes may be only a single layer thick or may be several layers thick. Moreover, the nanotubes may be carbon or other materials and may be both single wall and multi-wall nanotubes. This layer of nanotubes 82 may be provided on the exterior surface 83 of the catheter 81 in order to improve the lubricity of the catheter or some of its other external characteristics including the catheter's affinity for water.

Preferred medical devices for use in conjunction with the present invention include catheters, vascular catheters, balloon catheters, guide wires, balloons, filters (e.g., vena cava filters), vascular stents (including covered stents such as PTFE (poltetrafluoroethylene)-covered stents), stent grafts, cerebral stents, cerebral aneurysm filler coils (including GDC (Guglilmi detachable coils) and metal coils), vascular grafts, myocardial plugs, pacemakers, pacemaker leads, heart valves and intraluminal paving systems, filterwires, veinous valves, bifurcation stents, aortic stents and in essence all devices that can be utilized in the vascular system.

In addition to the embodiments described above, therapeutic may be delivered to the target directly upon the placement of the treated medical device at the target site through time-release from the nanotubes as they degrade over time.

The therapeutics that may be used are numerous and include pharmaceutically active compounds, nucleic acids with and without carrier vectors such as lipids, compacting agents (such as histones), viruses (such as adenovirus, andenoassociated virus, retrovirus, lentivirus and α-virus), polymers, hyaluronic acid, proteins, cells and the like, with or without targeting sequences.

Other examples of therapeutic agents used in conjunction with the present invention include, for example, pharmaceutically active compounds, proteins, cells, oligonucleotides, ribozymes, anti-sense oligonucleotides, DNA compacting agents, gene/vector systems (i.e., any vehicle that allows for the uptake and expression of nucleic acids), nucleic acids (including, for example, recombinant nucleic acids; naked DNA, cDNA, RNA; genomic DNA, cDNA or RNA in a non-infectious vector or in a viral vector and which further may have attached peptide targeting sequences; antisense nucleic acid (RNA or DNA); and DNA chimeras which include gene sequences and encoding for ferry proteins such as membrane translocating sequences (“MTS”) and herpes simplex virus-1 (“VP22”)), and viral, liposomes and cationic and anionic polymers and neutral polymers that are selected from a number of types depending on the desired application.

Non-limiting examples of virus vectors or vectors derived from viral sources include adenoviral vectors, herpes simplex vectors, papilloma vectors, adeno-associated vectors, retroviral vectors, and the like.

Non-limiting examples of biologically active solutes include anti-thrombogenic agents such as heparin, heparin derivatives, urokinase, and PPACK (dextrophenylalanine proline arginine chloromethylketone); antioxidants such as probucol and retinoic acid; angiogenic and anti-angiogenic agents and factors; anti-proliferative agents such as enoxaprin, angiopeptin, rapamycin, angiopeptin, monoclonal antibodies capable of blocking smooth muscle cell proliferation, hirudin, and acetylsalicylic acid; anti-inflarmatory agents such as dexamethasone, prednisolone, corticosterone, budesonide, estrogen, sulfasalazine, acetyl salicylic acid, and mesalamine; calcium entry blockers such as verapamil, diltiazem and nifedipine; antineoplastic/antiproliferative/anti-mitotic agents such as paclitaxel, 5-fluorouracil, methotrexate, doxorubicin, daunorubicin, cyclosporine, cisplatin, vinblastine, vincristine, epothilones, endostatin, angiostatin and thymidine kinase inhibitors; antimicrobials such as triclosan, cephalosporins, aminoglycosides, and nitrofurantoin; anesthetic agents such as lidocaine, bupivacaine, and ropivacaine; nitric oxide (NO) donors such as linsidornine, molsidomine, L-arginine, NO-protein adducts, NO-carbohydrate adducts, polymeric or oligomeric NO adducts; anti-coagulants such as D-Phe-Pro-Arg chloromethyl ketone, an RGD peptide-containing compound, heparin, antithrombin compounds, platelet receptor antagonists, anti-thrombin antibodies, anti-platelet receptor antibodies, enoxaparin, hirudin, Warfarin sodium, Dicumarol, aspirin, prostaglandin inhibitors, platelet inhibitors and tick antiplatelet factors; vascular cell growth promotors such as growth factors, growth factor receptor antagonists, transcriptional activators, and translational promotors; vascular cell growth inhibitors such as growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin; cholesterol-lowering agents; vasodilating agents; agents which interfere with endogenous vascoactive mechanisms; survival genes which protect against cell death, such as anti-apoptotic Bc1-2 family factors and Akt kinase; and combinations thereof. Cells can be of human origin (autologous or allogenic) or from an animal source (xenogeneic), genetically engineered if desired to deliver proteins of interest at the insertion site.

Polynucleotide sequences useful in practice of the invention include DNA or RNA sequences having a therapeutic effect after being taken up by a cell. Examples of therapeutic polynucleotides include anti-sense DNA and RNA; DNA coding for an anti-sense RNA; or DNA coding for tRNA or rRNA to replace defective or deficient endogenous molecules. The polynucleotides can also code for therapeutic proteins or polypeptides. A polypeptide is understood to be any translation product of a polynucleotide regardless of size, and whether glycosylated or not. Therapeutic proteins and polypeptides include as a primary example, those proteins or polypeptides that can compensate for defective or deficient species in an animal, or those that act through toxic effects to limit or remove harmful cells from the body. In addition, the polypeptides or proteins that can be injected, or whose DNA can be incorporated, include without limitation, angiogenic factors and other molecules competent to induce angiogenesis, including acidic and basic fibroblast growth factors, vascular endothelial growth factor, hif-1, epidermal growth factor, transforming growth factor α and β, platelet-derived endothelial growth factor, platelet-derived growth factor, tumor necrosis factor α, hepatocyte growth factor and insulin like growth factor; growth factors; cell cycle inhibitors including CDK inhibitors; anti-restenosis agents, including p15, p16, p18, p19, p21, p27, p53, p57, Rb, nFkB and E2F decoys, thymidine kinase (“TK”) and combinations thereof and other agents useful for interfering with cell proliferation, including agents for treating malignancies; and combinations thereof Still other useful factors, which can be provided as polypeptides or as DNA encoding these polypeptides, include monocyte chemoattractant protein (“MCP-1”), and the family of bone morphogenic proteins (“BMP's”). The known proteins include BMP-2, BMP-3, BMP-4, BMP-5, BMP-6 (Vgr-1), BMP-7 (OP-1), BMP-8, BMP-9, BMP-10, BMP-11, BMP-12, BMP-13, BMP-14, BMP-15, and BMP-16. Currently preferred BMP's are any of BMP-2, BMP-3, BMP-4, BMP-5, BMP-6 and BMP-7. These dimeric proteins can be provided as homodimers, heterodimers, or combinations thereof, alone or together with other molecules. Alternatively or, in addition, molecules capable of inducing an upstream or downstream effect of a BMP can be provided. Such molecules include any of the “hedgehog” proteins, or the DNA's encoding them.

Coatings used with the present invention may comprise various polymeric material/drug agent matrices. These may be formed, for example, by admixing a drug agent with a liquid polymer, in the absence of a solvent, to form a liquid polymer/drug agent mixture. Curing of the mixture typically occurs in-situ. To facilitate curing, a cross-linking or curing agent may be added to the mixture prior to application thereof. Addition of the cross-linking or curing agent to the polymer/drug agent liquid mixture must not occur too far in advance of the application of the mixture in order to avoid over-curing of the mixture prior to application thereof Curing may also occur in-situ by exposing the polymer/drug agent mixture, after application to the luminal surface, to radiation such as ultraviolet radiation or laser light, heat, or by contact with metabolic fluids such as water at the site where the mixture has been applied to the luminal surface. In coating systems employed in conjunction with the present invention, the polymeric material may be either bioabsorbable or biostable. Any of the polymers described herein that may be formulated as a liquid may be used to form the polymer/drug agent mixture.

The coatings used in the present invention may be hydrophilic or hydrophobic, and may be selected from the group consisting of polycarboxylic acids, cellulosic polymers, including cellulose acetate and cellulose nitrate, gelatin, polyvinylpyrrolidone, cross-linked polyvinylpyrrolidone, polyanhydrides including maleic anhydride polymers, polyamides, polyvinyl alcohols, copolymers of vinyl monomers such as EVA, polyvinyl ethers, polyvinyl aromatics, polyethylene oxides, glycosaminoglycans, polysaccharides, polyesters including polyethylene terephthalate, polyacrylamides, polyethers, polyether sulfone, polycarbonate, polyalkylenes including polypropylene, polyethylene and high molecular weight polyethylene, halogenated polyalkylenes including polytetrafluoroethylene, polyurethanes, polyorthoesters, proteins, polypeptides, silicones, siloxane polymers, polylactic acid, polyglycolic acid, polycaprolactone, polyhydroxybutyrate valerate and blends and copolymers thereof as well as other biodegradable, bioabsorbable and biostable polymers and copolymers. Coatings from polymer dispersions such as polyurethane dispersions (BAYHDROL®, etc.) and acrylic latex dispersions are also within the scope of the present invention. The polymer may be a protein polymer, fibrin, collagen and derivatives thereof, polysaccharides such as celluloses, starches, dextrans, alginates and derivatives of these polysaccharides, an extracellular matrix component, hyaluronic acid, or another biologic agent or a suitable mixture of any of these, for example. In one embodiment of the invention, the preferred polymer is polyacrylic acid, available as HYDROPLUS® (Boston Scientific Corporation, Natick, Mass.), and described in U.S. Pat. No. 5,091,205. U.S. Patent No. 5,091,205 describes medical devices coated with one or more polyisocyanates such that the devices become instantly lubricious when exposed to body fluids. In another preferred embodiment of the invention, the polymer is a copolymer of polylactic acid and polycaprolactone.

While various embodiments of the present invention have been described, other embodiments are also plausible. For instance the implant may be notched or grooved such that the nanotube treatment may be placed therein. These grooves or notches may then be covered, thereby creating individual vats or channels of nanotube treatment.

1. A medical apparatus comprising:
a medical device sized for insertion into a patient, the medical device having a first surface, and a second surface; and,
a plurality of nanotubes associated with the first surface of the medical device.

2. The medical apparatus of claim 1 further comprising:
a plurality of nanotubes associated with the second surface of the medical device.

3. The medical apparatus of claim 1 wherein the plurality of nanotubes associated with the first surface of the medical device is comprised of a single layer of nanotubes.

4. The medical apparatus of claim 1 wherein therapeutic is associated with the plurality of nanotubes.

5. The medical apparatus of claim 4 wherein the therapeutic is carried within the nanotubes of the plurality of nanotubes.

6. The medical apparatus of claim 4 wherein a portion of a molecule of the therapeutic is carried within a first nanotube from the plurality of nanotubes and the remainder of the molecule is positioned outside of the first nanotube from the plurality of nanotubes.

7. The medical apparatus of claim 1 wherein the plurality of nanotubes are positioned within a coating.

8. The medical apparatus of claim 4 wherein the therapeutic and the nanotubes are positioned within a coating.

9. The medical apparatus of claim 2 wherein the plurality of nanotubes associated with the second surface comprises more than one layer of nanotubes.

10. The medical apparatus of claim 1 wherein the medical device is either a stent or a catheter.

11. A method of treating a medical device sized for insertion into a patient, the method comprising:
providing a plurality of nanotubes for interfacing with the medical device; and
interfacing the plurality of nanotubes with the medical device.

12. The method of claim 11 further comprising:
interfacing the plurality of nanotubes with a therapeutic.

13. The method of claim 11 wherein the plurality of nanotubes form a layer of single nanotubes on the medical device.

14. The method of claim 11 wherein the plurality of nanotubes are within a carrier and wherein the plurality of nanotubes are associated with at least one therapeutic.

15. A method of treating target site comprising:
delivering a nanotube associated with at least one molecule of a therapeutic to a target site; and
breaking the nanotube in order to release one or more molecules of the thereapeutic.

16. The method of claim 15 wherein breaking the nanotube includes expanding a medical device associated with the nanotube.

17. A method of medical diagnosis comprising:
inserting a plurality of nanotubes into a body of a patient;
positioning the plurality of nanotubes at a target site within the body of the patient;
interfacing the plurality of nanotubes with the target site;
removing the plurality of nanotubes from the target site; and
analyzing the plurality of nanotubes after they have been removed from the target site.

18. The method of claim 17 wherein interfacing the plurality of nanotubes includes pressing the nanotubes against the target site and expanding a medical device carrying the nanotubes.

19. The method of claim 17 wherein analyzing the plurality of nanotubes includes analyzing the physical orientation of the nanotubes and analyzing material removed from the target site.

20. A method for manufacturing a medical device sized for insertion into the body, the system comprising:
providing a medical device; and
interfacing a medical device with a plurality of nanotubes.

21. The method of claim 20 further comprising:
dipping the medical device into a vessel containing a solution of nanotubes.

22. The method of claim 20 further comprising: rotating the medical device while it is being interfaced with the plurality of nanotubes.
